(19) United States
(12) Patent Application Publication
(10) Pub. No.: US 2025/0208117 A1
(43) Pub. Date: Jun. 26, 2025

(54) Superabsorbent Polymer based Biosensor Apparatus and Methods

(71) Applicant: IDE8 LIMITED, Inverness Highland (GB)

(72) Inventors: David HOWARTH, Inverness Highland (GB); Gavin MACFIE, Inverness Highland (GB); Andrew TREWEEKE, Inverness Highland (GB); Ian MEGSON, Inverness Highland (GB)

(21) Appl. No.: 18/848,788

(22) PCT Filed: Mar. 16, 2023
(86) PCT No.: PCT/GB2023/050623

§ 371 Date: Sep. 19, 2024

(30) Foreign Application Priority Data
Mar. 23, 2022 (GB) 2294051.3

(51) Int. Cl. G01N 33/52; G01N 21/77

(52) U.S. Cl. CPC G01N 33/52; G01N 21/77; G01N 2021/7759

(57) ABSTRACT

The invention relates to an absorbent sensing material for interrogation of sample liquid to detect analyte, comprising: dry superabsorbent polymer (SAP) configured to receive a sample liquid; the absorbent material comprising, within the dry superabsorbent polymer, at least one dry reaction component for detecting and for signalling the presence of 5 analyte; wherein, upon insult of sample liquid, the dry superabsorbent polymer absorbs sample liquid, the at least one dry reaction component dissolves and, if analyte is present in the sample liquid, its presence is detected and signalled. The invention also relates to use of the absorbent sensing material, absorbent articles and methods of manufactures of absorbent articles and test sensors such as test strips.

### FIELD OF THE INVENTION

The invention relates to an absorbent sensing material, which may be in particulate form, for interrogation of sample liquid to detect analyte, use of absorbent sensing material in absorbent articles, and in test sensors such as test strips, absorbent articles, test sensors such as test strips, and methods of manufacture of absorbent articles and test sensors such as test strips, as well as processes for detecting analyte in sample liquid using absorbent sensing material.

### BACKGROUND

Hydrogel-based Biosensors and Sensing Devices for Drug Delivery by Nicholas A. Peppas and Diana Snelling Van Blarcom, The Americas Special Issue of the Journal of Controlled Release 2016 describes glucose oxidase immobilized in a water swollen hydrogel for sensing glucose and swelling or de-swelling to provide insulin delivery in vivo. Stability of hydrogel sensing systems remains a problem.

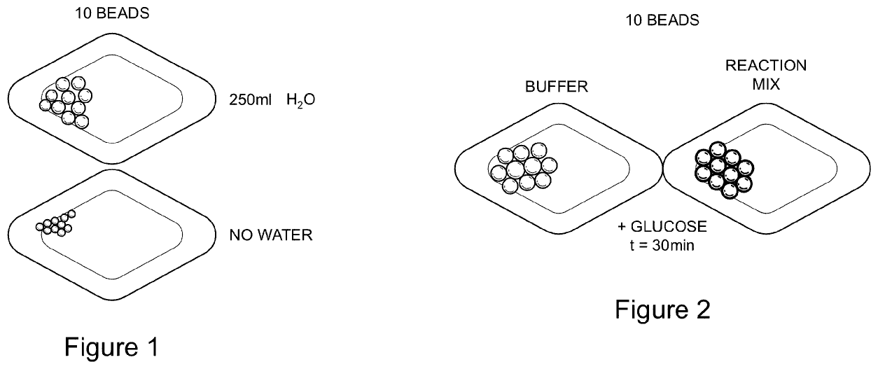

In Hydrogel Based Sensors for Biomedical Applications: An Updated Review, Polymers 2017, 9, 364, Javad Tavakoli and Youhong Tang comment on stability problems; various biologically-active components and transducing strategies are described (see FIG. 1). Table 2 describes strategies for small biomolecule detection using hydrogel-based biosensors for detection of glucose, lactate, urea and cholesterol. Such transduction strategies may be used in one or more embodiments of the present invention. It is of note that the authors conclude:

‘Some limitations, including life time, storage, and adaptation with transducers for rapid quantitative analysis reveal that there is still a long way for hydrogel-based biosensors to go before they are used in commercialized health management systems.’

Therefore, problems remain using hydrogel in quantitative and qualitative analysis. Cost and simplicity are also challenges in real life, commercial applications, particularly consumer applications.

In Superabsorbent Polymer Materials: A Review, Iranian Polymer Journal Vol 17 (6), 2008, 451-477, Mohammad J. Zohuriaan-Mehr and Kourosh Kabiri, describe superabsorbent polymers and their preparation which may be used in one or more embodiments of the present invention.

Biomedical applications of hydrogels: A review of patents and commercial products, Enrica Caló, Vitaliy V. Khutoryanskiy, in European Polymer Journal 65 (2015) 252-267 describes various applications including contact lenses, wound dressings, drug delivery, tissue engineering and hygiene products. The authors describe ‘swelling-controlled release devices’, seemingly for in vivo use.

Enzyme-Responsive Polymer Hydrogel Particles for Controlled Release, Thornton et al, 2007—Advanced Materials 19, Issue 9 describes a swelling response in hydrogel particles releasing physically entrapped macro-molecules for drug delivery in vivo.

Absorbent articles such as diapers comprising biosensing capabilities are known. For example, KR20180085577 LEE describes an attachment-type sensor comprising first and second hydrogel membranes and adhesive film. The flow of the sample does not directly affect the test strip, so the reaction agent is less likely to be washed off. Similar to LEE, WO00/65348 ROE (Proctor and Gamble) describes disposable articles comprising a detection device including a discrete detection device such as a biosensor. US2015025347 SONG describes a separate lateral flow device attached to an absorbent article. WO2015085024 NELSON describes a disposable hygienic article with means for diagnostic testing stating typically in the form of a separate test strip visible through a port. At paragraph 37 its states, ‘while some literature indicates that the presence of SAP in a diaper used for urine testing is considered benign, manufacturing diapers without SAP for the benefit of accuracy is contemplated by the present invention and may be considered beneficial’. Thus, the use of SAP is not considered beneficial when detecting disease. Further, such discrete detection devices or test strips may break or detach and are costly and complex to manufacture in addition to the disposable article.

U.S. Pat. No. 5,468,236 EVERHART describes a disposable product incorporating a chemically reactive substance to provide a visual indication and states discharges of additional substances will have little effect on the visual indication provided by the chemically reactive means, but does not appear to describe how. In any case, this appears complex and costly to manufacture.

WO2015/127528 JOLLEZ describes a chromogenic absorbent material for animal litter comprising a water-absorbing first polysaccharide, and a structural second polysaccharide. Similarly, WO2017/165953 JOLLEZ describes a water absorbing material and uses thereof.

U.S. Pat. No. 5,200,321 KIDWELL describes a micro assay on a card with various layers. JPH11295306 BISEIKEN describes test materials for urine inspection using cellulose. JP2017187336A KOGA SHIN describes a urinary ketone body detecting paper diaper with an indicator embedded between and absorbent body and a transparent window.

WO2018/141017 RAJAPAKSA describes an absorbent article with indicator. Bilirubin oxidase catalyses a colorimetric reaction visible when urine with bilirubin is present. A separate absorbent material is used within the absorbent body capable of collecting and retaining urine. The bilirubin oxidase may be encapsulated in a micro-capsule such as polysaccharide or the bilirubin oxidase and may be stabilised by covalently binding to polyacrylic acid. Neither is an easy to manufacture or cost-effective route to long term stability.

US2006/0253047 FOX describes a device for non-invasively monitoring a medical condition of a mammal and in particular marker ingredients to generate a colour in a non-invasive wearable device for a collecting bodily fluid. Several options for marking ingredient application are described, but application in a liquid carrier to the inner or outer surface of the next-to-skin water permeable layer is preferred.

WO2016191372 MEEK describes an indicator panel which may be in contact with an absorbent core. The problem of highly concentrated urine affecting perceived indicator colour is noted. WO2008/072116 LONG is similar to MEEK describing absorbent articles that can detect medical conditions.

US2015260658 SONG describes multi-layered devices for analyte collection using a control layer to inhibit the reaction at a sensing layer and acknowledges problems of repeated insults of bodily fluid. A separate absorption layer is required.

US2017/0172933 HARDER describes loadable polymeric particles for therapeutic and diagnostic applications.

Diaper Detective System 2014 is a diaper with an insert lateral flow channels for two biosensing tests. This is complex to manufacture.

WO2007073139 and MXNL05000103 GONZALEZ describe a diagnostic disposable diaper including: a plurality of urine-sensitive indicator reagents to detect levels of glucose, ketone bodies, heavy metals and which are fixed to the internal absorbent lining of the diaper.

SHIBATA et. Al. 17894-17898 |PNAS| Oct. 19, 2010 describes injectable hydrogel microbeads for fluorescence based in vivo continuous glucose monitoring. WO2012043177 (EP2623530) SHIBATA describes injectable hydrogel particles.

U.S. Pat. No. 6,203,496 GAEL describes providing one or more chemical reagents near an absorbent region of a diaper and examples of various detection regimes, reagents and colour changes that may be useful in embodiments of the present invention.

In Simultaneous detection of pH value and glucose concentrations for wound monitoring applications, JANKOWSKA et al. describe a wound pad for non-invasive monitoring using sensing molecules in a biocompatible hydrogel matrix.

WO2019245468 SISMAN describes a method of manufacture to introduce chemicals to non-woven bulk product for diagnosing a disease.

Various non-biosensing applications are known. Non-biosensing applications include treatment. No signalling component is required or provided, or in several examples, possible.

U.S. Pat. No. 6,051,749(A) SCHULZ describes a fabric incorporating organophilic clay, preferably dispersed in a matrix of a superabsorbent polymer for diapers that can prevent skin irritation.

US2012/0058074 BRAIG describes dry odour-inhibiting compositions comprising water absorbing polymer particles and at least one oxidase and a substrate of the oxidase e.g. β-D-Glucose for glucose oxidase.

KR930002270(B1) IM describes disposable absorbents with anti-bacterial enzyme.

US 2013/0345655 WU describes a dried polymeric matrix with embedded enzyme for de-odour applications.

WO2019/077335 HALL describes antimicrobial super-absorption compositions comprising an enzyme, a substrate for the enzyme, and a superabsorbent component, such as a superabsorbent polymer, in the form of a powder.

WO2013116358 STAMPER describes insecticidal hydrogel feeding spheres.

US2017348162 ARIZTI describes an absorbent device and a detector device to indicate the presence or absence of bodily oxidates in an absorbent system but does not discuss disease detection.

U.S. Pat. No. 5,226,902 BAE describes a device for dispensing biologically active material into the surrounding environment in which hydrogel deswells or shrinks in response to contact by external physical or chemical stimuli.

WO2017178417A1 DONG describes swelling of polymer beads to close an inlet to a sample chamber.

Su, Xin et al describe Hydrophilic/hydrophobic heterogeneity anti-biofouling hydrogels with well-regulated rehydration in: ACS Applied Materials and Interfaces. 2020; Vol. 12, No. 22. Pp. 25316-25323.

Ye, B.-F. et al describe a Colorimetric photonic hydrogel aptasensor for the screening of heavy metal ions in Nanoscale, 4(19), 5998 (2012).

Liu, H. et al describe Enzyme encapsulation in freeze-dried bionanocomposites prepared from chitosan and xanthan gum blend. Materials Chemistry and Physics, 129(1-2), 488-494 (2011).

Zhang, Z. et al describe Encapsulation of lactase (β-galactosidase) into κ-carrageenan-based hydrogel beads: Impact of environmental conditions on enzyme activity. Food Chemistry, 200, 69-75 (2016).

Wolf, M. et al describe Stability of β-D-galactosidase immobilized in polysaccharide-based hydrogels. Colloids and Surfaces A: Physicochemical and Engineering Aspects, 609, 125679 (2021).

Ghanem, A., & Ghaly, A. describe Immobilization of glucose oxidase in chitosan gel beads. Journal of Applied Polymer Science, 91(2), 861-866 (2003).

### Problems Remain with Respect to the Art

the concentration of analyte in sample liquid, e.g. urine, may vary with time;
when using wearable articles, e.g. diapers, to mount a separate test element there is a variable flow, e.g. of urine, and therefore a variable contact time, and uncontrolled, variable exposure and/or development time;
additional insults of liquid sample can wash away and/or dilute reaction components, particularly in known configurations.
where marker ingredients are placed alongside absorbent cores, in say wearable articles, additional insults of bodily fluid can dilute and/or sweep away the developed (indicating) marker ingredients;
sample fluid discharged into an absorbent matrix increases in concentration as water is selectively absorbed into an absorbent core, affecting the remaining liquid presented to any test element;
test strip sensors attached to diapers may be configured to limit volume to address some of these issues but may suffer from other issues, such as cost and complexity of manufacture and use;
test strips or other biosensor components placed on or attached to wearable articles e.g. diapers, are often rigid and may break (leaking fluid) and/or be uncomfortable to wear, they also add significantly to cost:
providing enzyme substrates e.g. glucose for glucose oxidase prevents the detection of such substrates;
where drug delivery is envisaged, a surrounding medium that provides a diffusion gradient is required, this is undesirable in many commercial applications;
hydrogel-sensing systems degrade relatively quickly and are unstable over the time periods for long term storage required for commercial products;
defining a volume for liquid sample can be a challenge in manufacturing
complex structures with layers are difficult to manufacture and control, and may require pre-processing of analyte before a conjugate or the like is received in an absorbent layer.

The present invention seeks to alleviate one or more of the above problems, and/or other problems of the prior art.

### Statements of the Invention

In a first aspect of the invention there is provided, an (e.g. dry) absorbent sensing material for interrogation of sample liquid to detect analyte (e.g. the presence and/or concentration of analyte) (e.g. in an absorbent article),
(preferably) comprising (or more preferably substantially consisting of, or more preferably essentially consisting of, or more preferably consisting of, or more preferably only consisting of):
dry superabsorbent polymer (SAP) (e.g. dry super absorbent polymer material which may be known as modified e.g. pre-loaded dry superabsorbent polymer) configured to receive a sample liquid (e.g. in vitro);
the absorbent material comprising, within the (modified e.g. pre-loaded) dry superabsorbent polymer, (e.g. dried within the superabsorbent polymer to form modified,
e.g. pre-loaded, dry superabsorbent polymer material) at least one dry reaction component for (e.g. configured for) detecting and for signalling the presence of analyte (e.g. in vitro);
wherein, upon insult of sample liquid, the dry (modified) superabsorbent polymer absorbs sample liquid, the at least one dry reaction component dissolves and, if analyte is present in the sample liquid, its presence is detected and signalled (e.g. within the SAP, from within the SAP).

In at least one embodiment, the dry superabsorbent polymer (SAP) (which may be known as modified, e.g. pre-loaded, dry superabsorbent polymer) changes colour and/or fluoresces.

The absorbent sensing material preferably comprises at least 50%, more preferably at least 75%, more preferably at least 90%, more preferably at least 95% by weight of dry superabsorbent polymer comprising the at least one dry reaction component.

The (modified) dry superabsorbent polymer material preferably comprises at least 95% by weight, more preferably at least 97% by weight of dry superabsorbent polymer and may comprise up to 5% by weight, more preferably 3% by weight, of one or more dry reaction component(s).

Preferably at least one dry first reaction component is provided for detecting analyte and at least one dry second reaction component is provided for signalling the presence of the detected analyte.

Preferably, the (modified) dry superabsorbent polymer (SAP) is configured to receive a discrete (e.g. limited) amount of a sample liquid (e.g. in vitro); Preferably, the (modified) dry superabsorbent polymer provides (e.g. defines) a sample chamber.

Preferably, the (modified) dry superabsorbent polymer provides (e.g. defines) a sample chamber of limited mass.

Preferably, the (modified) dry superabsorbent polymer provides (e.g. defines) a sample chamber of limited volume (e.g. in a dry state and/or in a swollen hydrated state).

Preferably, the (modified) dry superabsorbent polymer is in the form of a continuous bulk medium, having a discrete (e.g. limited) absorption capacity per unit mass providing, in effect, sample chamber of limited volume per unit mass.

Preferably, the (modified) dry superabsorbent polymer comprises particles, preferably dry, preferably discrete particles. In this way the SAP particles each provide a discrete quantity of SAP into which a limited, albeit large, volume of liquid can be absorbed. The SAP is preferably formed as particles (e.g. beads, pellets, flakes, grains of any shape e.g. spherical, cylindrical) of small mass and so (relatively) small swelling capability. SAP particles do tend to have very rapid and large swelling capability although this slows very quickly following after initial exposure to liquid. The SAP particles are preferably 3 dimensional (3D) although they may be generally 2 dimensional, and/or have limited size in one dimension e.g. flakes. They may be hollow. In one or more embodiments it is the provision of discrete particles of dry SAP in combination with one or more dry detection and signalling components pre-loaded within the SAP that provides one or more advantages of the invention. Thus, the (modified) dry SAP of the invention may also be referred to as pre-loaded dry SAP.

Preferably, the dry particles are of similar mass e.g. at least 90% by weight are within a range of ≤+/−10% the average dry mass. Preferably, at least 90% by weight of the particles are within ≤±10% or ≤±5% or ≤±2% of the average dry weight of the particles. For example, so 1 mm size particles (e.g. beads) have approximately 200 particles (e.g. beads) per gram. 2.5 mm diameter particles (e.g. beads) may have approximately 65 particles per gram. 4 mm diameters particles (e.g. beads) may have approximately 20 particles per gram.

Preferably, the particles are of similar size. Preferably, at least 90% by weight of the particles are within ≤±10% or ≤±5% or ≤±2% of the average dimension (e.g. length or diameter or radius). When dry, the particles may be of the order of tens or hundreds of nanometres e.g. submicron so for example ≤100 nanometres, or 100 to 1000 nanometres, or may span the nanometre and micron range so for example 100 to 5000 nanometres, or may be of order of tens or hundreds of microns, so submillimetre e.g. from 5 to 1000 microns, or 5 to 500 microns, 5 to 250 microns, or ≤500 microns, or above the half millimetre range so e.g. 0.5 to 5 mm, or within the millimetre range from 1 mm to 4 mm or from 0.5 mm to 3 mm, or from 0.5 mm to 2 mm, or from 1 mm to 2 mm in main lateral dimension e.g. length or diameter (so, for example, 90% of particles may have these dimensions, e.g. within +/−10% or +/−0.1 mm or +/−0.2 mm). Being of substantially or generally consistent density, each SAP particle may therefore have substantially or generally the same mass and so the same liquid absorption capacity. Thus, the absorptive capacity per unit mass, or per particle, defines a ‘sample chamber’.

This may be particularly useful when the dimension is known. In one or more embodiments, for example, a main dimension of dry SAP particles, such as length or diameter, preferably an average main dimension over a known number of particles, is, preferably ≤100 nanometres, or preferably 100 to 1000 nanometres, or preferably 100 to 5000 nanometres, 5 to 1000 microns, 5 to 500 microns, 5 to 250 microns, ≤500 microns or preferably ≤0.1 mm (100 micrometres), or preferably ≤0.05 mm (≤50 micrometres), or preferably ≤0.01 mm (≤10 micrometres), or preferably ≤0.005 mm (≤5 micrometres), 0.5 to 5 mm, or from 1 mm to 4 mm, or from 0.5 mm to 3 mm, or from 0.5 mm to 2 mm, or from 1 mm to 2 mm. Preferably, for example, at least 50%, or at least 80%, at least 85%, or at least 90%, or at least 95% or at least 99% of particles have such a corresponding main dimension e.g. within +/−10% or within +/−5%.

The purpose of selecting such a small main dimension (for example for the majority such as ≥50%, or ≥75%, or ≥90%, or ≥95%, in other words, preferably most) for SAP particles is so that the volume of the sample chamber provided by each particle is more easily delimited or defined to be of particular size or of a particular range of sizes as described elsewhere herein. The effective size of the sample chamber from one particle to the next is preferably similar or the same within a similar range of tolerances.

Preferably, in use or when used in an absorbent article for a particular use, the limited volume (e.g. per particle) is less than an expected volume of sample liquid. Preferably, the limited volume (e.g. per particle) is at least an order of magnitude less than an expected volume of sample liquid.

Preferably, the at least one dry reaction component comprises
at least one dry transducing component for detecting an analyte;
and,
at least one dry signalling component responsive to the detection of analyte for signalling the presence of analyte.

Preferably, the at least one reaction component or the at least one transducing component comprises at least one of
an electrochemical assay;
a colorimetric assay;
a nitrate assay;
a ketone assay;
a glucose assay;
a bilirubin assay;
a component capable of photometric detection;
a component capable of spectrometric detection.

Preferably, the at least one reaction component or the at least one transducing component comprises a colorimetric assay comprising a chromogen (and/or dye).

Preferably, the at least one dry transducing component is selected from:
one or more enzymes;
Glucose Oxidase (GOD);
Glucose Oxidase (GOD) and Horseradish Peroxidase (HRP)
and the at least one signalling component is selected from
a chromogen
a colourless chromogen
potassium iodide (e.g. to provide brown colours)
3, 3′, 5, 5′ tetramethylbenzidine (TMB) (e.g. to provide blue colours). In one or more example embodiments, for example, a Benedict's solution will change from a transparent blue colour to orange or brick red precipitate depending on the level of glucose present, whereas green represents normal glucose.

Preferably, the superabsorbent polymer is a synthetic superabsorbent polymer, or a naturally-derived superabsorbent polymer, or a biodegradable superabsorbent polymer, or any combination thereof.

In one or more embodiments the superabsorbent polymer comprises, or consists of, one or more SAPs recognised or determined by international standards such as: ISO 17190:2019 which refers to ‘Polymeric material capable of absorbing and retaining large quantities of aqueous solutions, saline solutions, and body fluids’ or ISO 12687:2019 which refers to ‘Polymeric materials, based on acrylic acid, acrylamide or their derivatives, that can absorb and retain water and aqueous solutions to a degree that the resulting hydrogel products can absorb at least 10 times their own weight of water under pressure’ or ASTM F2221-15 which refers to ‘polymeric material that, when hydrated, swells and retains large amounts of liquid, such as water or aqueous solutions, as a result of the action of capillary forces or hydrogen bonding or both.’

Preferably, the superabsorbent polymer comprises at least one of:
polymerised acrylic acid or salts thereof or the like, sodium polyacrylate, polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile, e.g. at least 50% or at least 75% or at least 90% by weight of one or more of these.

In a further aspect, there is provided a disposable absorbent article for sample (e.g. bodily) fluid interrogation (e.g. urine, interstitial fluid, tears, saliva, plasma, serum, blood etc) comprising an absorbent core, at least a portion of the absorbent core comprising the absorbent material of any of claims 1 to 20 or as described.

Preferably, in use or when used in an absorbent article for a particular use, the limited volume (e.g. per particle) is less than an expected volume of sample liquid. Preferably, the limited volume (e.g. per particle) is at least an order of magnitude less than an expected volume of sample liquid.

Preferably, at least a portion of the (modified e.g. pre-loaded) dry superabsorbent polymer (SAP) of the absorbent material comprises particles of (modified e.g. pre-loaded) superabsorbent polymer (SAP).

Preferably, at least a portion of the dry superabsorbent polymer (SAP) of the absorbent material comprises superabsorbent polymer (SAP) fibres e.g. to provide or be added to fibres in, for example, absorbent sheets, such as nappy liners, hygiene liners, kitchen roll, toilet roll, tissues etc.

Preferably, the absorbent material is provided within a compartment of limited volume, having at least one impervious wall (e.g. a rear wall, or rear and one or both side walls) to resist further ingress of sample liquid once full.

In a further aspect, there is provided use of an absorbent sensing material according to any of claims 1 to 20 or as described herein for detecting and for signalling the presence of one or more analytes in a sample liquid (e.g. in vitro), preferably in which the superabsorbent polymer is used as a sample chamber to absorb a predetermined amount of the at least one reaction component, and/or preferably in which the superabsorbent polymer is used as a sample chamber to absorb a predetermined amount of sample liquid.

In a further aspect, there is provided method of manufacturing an absorbent sensing material according to any of claims 1 to 20, comprising:
preparing SAP (e.g. particles of a predetermined dimension or range of dimensions e.g. volume or length or diameter to provide, when swollen, a predetermined swollen volume or range of swollen volumes), optionally, of known or predictable size or range of sizes as discussed in various example herein; drying SAP particles;
in the absence of an analyte, introducing at least one wet reaction component for detecting and signalling the presence of the analyte, to the dry SAP;
drying the SAP to form dry SAP pre-loaded with the at least one dry reaction component for detecting and signalling the presence of an analyte.

In a further aspect, there is provided a method of manufacturing an absorbent sensing material according to any of the claims 1 to 20 or as described herein, comprising:
preparing SAP particles;
drying SAP particles;
in the absence of an analyte, introducing at least one wet reaction component for detecting and signalling the presence of the analyte, to the dry SAP particles; drying the SAP particles to form dry SAP particles pre-loaded with the at least one dry reaction component for detecting and signalling the presence of an analyte.

In a further aspect, there is provided a process for detecting analyte in a fluid (e.g. in vitro) comprising:
a) providing absorbent material according to any of the claims to absorbent material 1 to 20, or as described herein, or an absorbent article according to any of claims to absorbent articles, or as described herein;
b) insulting the absorbent sensing material comprising (modified e.g. pre-loaded) dry SAP, preferably (modified e.g. pre-loaded) dry SAP particles, with sample liquid;
c) optionally, re-insulting the absorbent sensing material with sample liquid (e.g. fresh sample liquid);
d) at least partially rehydrating the initially dry (modified e.g. pre-loaded) SAP;
e) at least partially rehydrating the at least one reaction component (e.g. within the SAP) to detect and signal the presence (and/or concentration) of analyte in the sample liquid;
f) detecting and signalling the presence of analyte in the sample liquid (e.g. by visual indication within the SAP such as a change of colour of the SAP).

In one aspect, the invention provides dry SAP beads encapsulating a detection chemistry that activates within the limited volume of the beads upon intake of a liquid, e.g. biological fluid in an absorbent product, to provide a visible result and methods of manufacturing and uses of same.

Several embodiments of the invention are described and any one or more features of any one or more embodiments may be used in any one or more aspects of the invention as described herein as would be understood by someone skilled in the art. Thus, the features of the claims and/or those described herein may be used in any aspect of the invention, the different aspects of the invention making use of the features of embodiments described with reference to a different aspect. For example, whilst SAP particles and in particular SAP beads are described in several examples below, it will be understood that technical features of the SAP particles and in particular the SAP may be adapted to SAP in non-particulate form or in other shapes (e.g. SAP bulk medium, and/or SAP fibres etc.) with suitable changes, mutatis mutandis, as would be understood by someone skilled in the art.

### BRIEF DESCRIPTION OF THE INVENTION

The present invention will now be described, by way of example only, with reference to the following figures.

FIG. 1 shows a perspective view of 10 SAP beads when initially dry (lower) and when completely swollen (upper).

FIG. 2 shows a perspective view of groups of 10 SAP beads in Experiment 1 with beads pre-treated (hydrated) with buffer only (left) and beads pre-treated (hydrated) with liquid reaction mix (right)

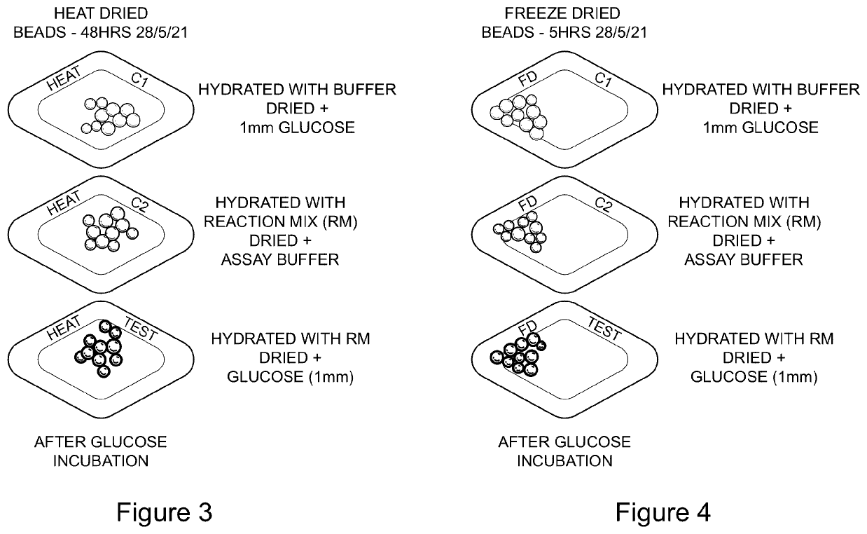

FIG. 3 and FIG. 4 show perspective view of groups of 10 SAP beads in Experiments 2 and 3 showing respectively heat-dried beads (FIG. 3) and freeze-dried beads (FIG. 4).

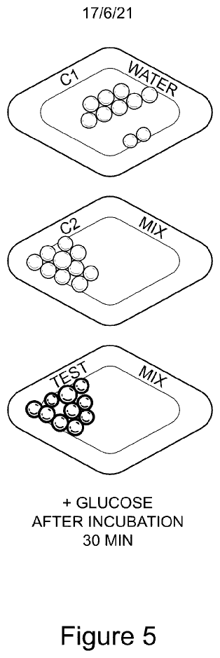

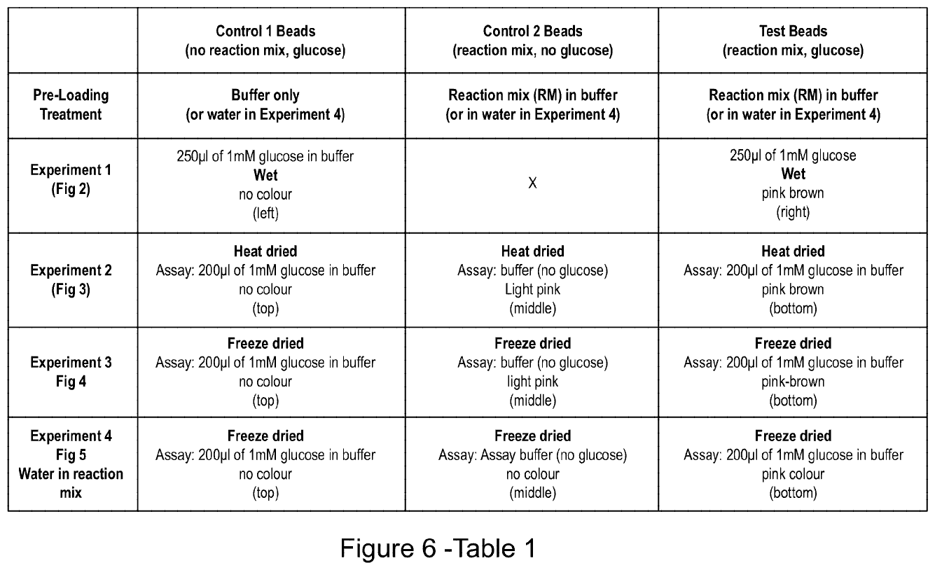

FIG. 5 shows perspective view of groups of 10 SAP beads in Experiment 4.

FIG. 6 is a table of results.

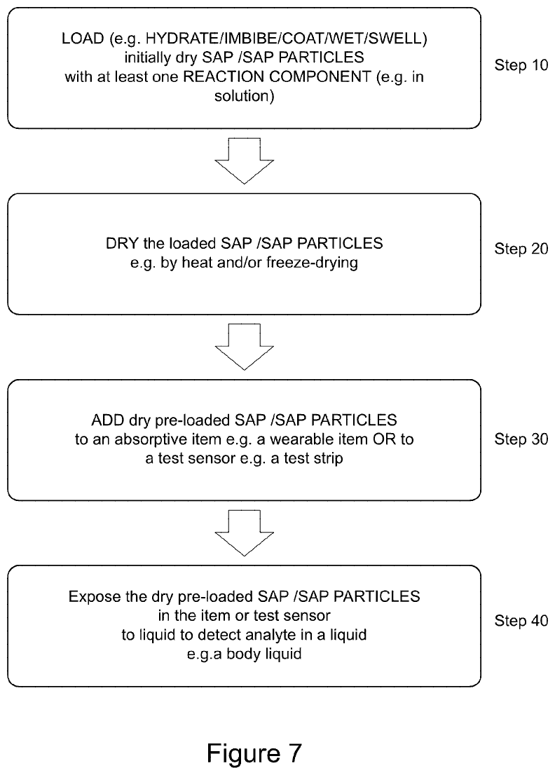

FIG. 7 is a flow chart outlining one or more processes of the invention.

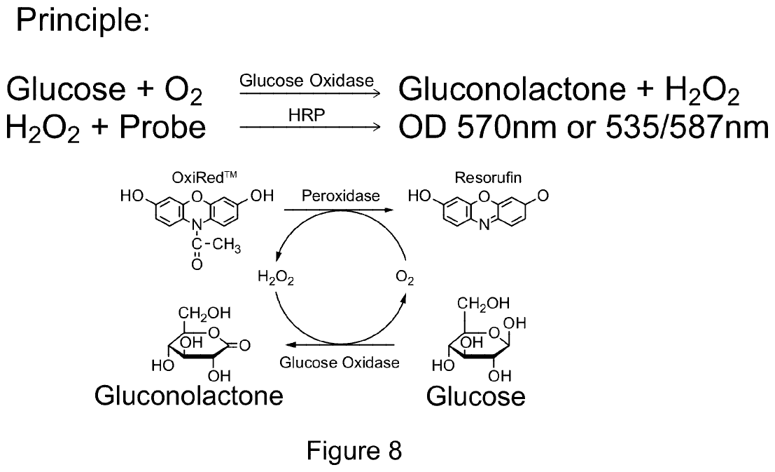

FIG. 8 is an example chemical detection and signalling regime for detection of glucose in one or more example embodiments of any aspect of the invention.

### DETAILED DESCRIPTION OF THE INVENTION

Any quantities, dimensions, or shapes used herein are to be understood to lie within the tolerances, ranges, and variations expected or understood within the particular field. For example, beads are usually spherical but need not be so, and spherical beads may vary in their sphericity. In this disclosure, the term ‘beads’ is used as one example of the more general term ‘particles’, which are discrete, independently movable from one another, each having a limited mass, which may vary from one to the next. Further within the following description SAP may refer to SAP alone, or, where the context so indicates, to modified SAP material comprising both SAP and at least one reaction component and the present text is to be read with this in mind. SAPs are defined in several standards including ISO standards referred to elsewhere in this text.

Hydrogel Biosensors have generated considerable interest over the past few years, with respect to the excellent alternative systems to detect a wide range of biomolecules, including small biochemicals, pathogenic proteins, and disease-specific genes.

Due to the excellent physical properties of hydrogels, such as the high water-content and stimuli-responsive behavior of cross-linked network structures, this system can offer substantial improvement for the design of novel detection systems for various diagnostic applications. The other main advantage of hydrogels is the role of biomimetic three-dimensional (3D) matrix immobilizing enzymes and aptamers within the detection systems, which enhances their stability.

This provides ideal reaction conditions for enzymes and aptamers to interact with substrates within the aqueous environment of the hydrogel.

In comparison, a super absorbent polymer (SAP) is a hydrogel without the water content. In other words, a SAP is dry. SAP comprises water-absorbing polymers which when cross-linked, absorb aqueous solutions through hydrogen bonding with water molecules. A SAP's ability to absorb water, liquid or a biological fluid depends on the ionic concentration of the aqueous solution.

In this disclosure, a SAP (superabsorbent polymer) is intended to refer to such a polymer in dry form. This may be entirely dry (H₂O ≤0.5% by weight), essentially dry (55% H₂O by weight), substantially dry (≤15% H₂O by weight), or sufficiently dry (e.g. 520% H₂O by weight) such that the stability of the SAP and its contents (such as its reaction components) do not degrade to any significant extent over the period of preferably 6 months, more preferably 1 year, even more preferably 18 months, even more preferably 2 years. It is preferred that the SAP does not comprise enough or sufficient water to enable the transducer component e.g. enzyme to activate or to activate the signalling component.

Further, in this disclosure, a hydrogel is intended to refer to a SAP (superabsorbent polymer) in wet form. This may be partially swollen with liquid e.g. (≥25% H₂O by weight), or substantially swollen with liquid e.g. (≥50% H₂O by weight or ≥60% H₂O by weight or ≥70% H₂O by weight) or sufficiently swollen with liquid e.g. H₂O that further absorption of liquid is substantially reduced and/or prevented e.g. within the timescale of exposure to liquid.

In deionized and distilled water, a SAP may absorb 300 times its weight (from 30 to 60 times its own volume), but when put into a 0.9% saline solution, the absorbency may drop to 50 times its weight. Of note is the swift initial swelling in the first 200 seconds and the much slower absorption thereafter (see Zohuriaan-Mehr et al 2008). The present inventors have appreciated that this variation in speed of swelling can be used in short-lived disposable hygiene absorption products or interrogation mechanisms in sensors such as test sensors e.g. test strips to deliver an indicator of the presence of an analyte or of a disease or state or, indeed in some embodiments, a test result. Examples of analytes include but are not limited to glucose, lactate, urea and cholesterol.

Biosensor systems typically comprise:
i. a biologically active receptor element e.g. enzymes, optionally with one or more co-factor(s), antibodies, antigens, cells, nucleic acids, DNA etc.;
ii. a base material which may be a base substrate in the form of a layer of polymer or paper or other suitable material;
iii. a transducer element based on, for example, optical signalling (e.g. colour change, absorbance, luminescence, electrochemical (e.g. amperometric, conductometric, potentiometric, impedimetric etc.;
iv. a detection or observation element.

In the remainder of this document, we shall talk about glucose and glucose oxidase in a colorimetric system as examples of an analyte/receptor system with a transducer capability, but it will be understood that in other embodiments, other biosensor chemistries can be used in the different aspects of the invention.

Unlike the prior art, in this invention it is the absorptive material itself, the superabsorbent polymer, that provides the ‘vessel’ or ‘sample chamber’ within which the reaction takes place between the biologically-active receptor and an analyte and, preferably also, within which the visual signalling occurs. This combination of the dry biologically-active receptor and dry transducer element within a single dry component (the superabsorbent polymer) greatly simplifies the manufacture of wearable products and, indeed, biosensors which provide a disease state indication or, indeed, measurement and is particularly useful for wearable hygiene products such as diapers, sanitary napkins and, indeed, non-wearable hygiene products such as absorptive sheets for beds.

The approach of the present inventors is to use the properties of a SAP and combine this with a number of known bio reagents so that, when presented with a biological fluid sample, the super absorbent reagent will, firstly, capture and contain the biological sample and, secondly, will react to provide an indication of the presence or concentration of the analyte e.g. of elevated glucose or ketones. The indication may be directly observable e.g. visible by eye such as a change in colour, or may be provided after interrogation e.g. direct or indirect interrogation.

Industry-recognized assay techniques and reagent chemicals may be combined with SAP to provide this new approach.

Superabsorbent polymers are commonly made from the polymerization of acrylic acid blended with sodium hydroxide in the presence of an initiator to form a poly-acrylic acid sodium salt (sometimes referred to as sodium polyacrylate). This polymer is the most common type of SAP made in the world today.

Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile to name a few. The latter is one of the oldest SAP forms created (see CALO et al 2015).

Today, superabsorbent polymers are made using one of three primary methods: gel polymerization, suspension polymerization or solution polymerization. Each of the processes has their respective advantages but all yield a consistent quality of the product.

Gel polymerization—A mixture of acrylic acid, water, cross-linking agents and UV initiator chemicals are blended and placed either on a moving belt or in large tubs. The liquid mixture then goes into a ‘reactor’ which is a long chamber with a series of strong UV lights. The UV radiation drives the polymerization and cross-linking reactions. The resulting ‘logs’ are sticky gels containing 60-70% water. The logs are shredded or ground and placed in various sorts of driers. Additional cross-linking agent may be sprayed on the particles' surface; this ‘surface cross-linking’ increases the product's ability to swell under pressure—a property measured as Absorbency Under Load (AUL) or Absorbency Against Pressure (AAP). The dried polymer particles are then screened for proper particle size distribution and packaging. The gel polymerization (GP) method is currently the most popular method for making the sodium polyacrylate superabsorbent polymers now used in baby diapers and other disposable hygienic articles.

Solution polymerization—Solution polymers offer the absorbency of a granular polymer supplied in solution form. Solutions can be diluted with water prior to application, and can coat most substrates or used to saturate them. After drying at a specific temperature for a specific time, the result is a coated substrate with super absorbency. For example, this chemistry can be applied directly onto wires and cables, though it is especially optimized for use on components such as rolled goods or sheeted substrates.

Solution-based polymerization is commonly used today for SAP manufacture of copolymers, particularly those with the toxic acrylamide monomer. This process is efficient and generally has a lower capital cost base. The solution process uses a water-based monomer solution to produce a mass of reactant polymerized gel. The polymerization's own exothermic reaction energy is used to drive much of the process, helping reduce manufacturing cost. The reactant polymer gel is then chopped, dried and ground to its final granule size. Any treatments to enhance performance characteristics of the SAP are usually accomplished after the final granule size is created.

Suspension polymerization—The suspension process is practiced by only a few companies because it requires a higher degree of production control and product engineering during the polymerization step. This process suspends the water-based reactant in a hydrocarbon-based solvent. The net result is that the suspension polymerization creates the primary polymer particle in the reactor rather than mechanically in post-reaction stages. Performance enhancements can also be made during, or just after, the reaction stage.

In one or more embodiments, the invention provides a system in which a biological fluid is diffused into a SAP (e.g. into initially dry) SAP material, and more preferably into (e.g. initially dry) SAP particles, which has/have been bonded with a wide variety of chemical reagents during or after manufacture e.g. as outlined in any of the three types of SAP manufacture above. Thus, an initially dry SAP pre-loaded with one or more reaction components can be provided.

Thus, in one or more embodiments, the invention provides SAP+Reagent which when insulted by a sample liquid e.g., a biological fluid provides a hydrogel sample for interrogation. The biological sample acts as the wetting material within the superabsorbent polymer.

The invention aims to solve and improve upon methods of capturing a biological sample within a specific mechanical carrier (e.g. microfluidic or nanofluidic cell, paper-based assay/dipstick) to measure including small biochemicals, pathogenic proteins, and disease-specific genes.

One or more embodiments of the invention allow for diffusion of the specific sample that is under interrogation and allows for the act of absorption to mix the biological sample with the predefined reagent.

SAPs, once swollen with liquid, particularly once swollen to near or final capacity, are resistant to pneumatic compression, such as might occur in a sanitary article for example a female hygiene pad or diaper for a child or adult, or indeed an absorbent incontinence sheet for use in beds. The present inventors have recognised the usefulness of this capability in developing embodiments of the present invention.

Whereas typical absorbent materials such as wood or paper fibres can be ‘wrung out’ by applying pressure when wet. This does not occur to any significant extent in swollen SAP. Indeed, this is even less the case with swollen SAP particles which, even in a contained volume of a wearable article or incontinence sheet, the particles can move a little with respect to one another, alleviating pressure. Thus, liquid, once absorbed by SAP particles, remains absorbed. Furthermore, SAPs, once swollen with liquid, even partially, demonstrate a far lower speed of absorption e.g. swelling rate. The present inventors have recognised the usefulness of this capability.

Furthermore, bearing in mind SAP particles have a limited mass, they therefore also have a limited, although great, ability to absorb liquid. So, in this sense, they have a limited volume, a limited volume into which sample can be absorbed. This may be thought of as a sample chamber. Thus, once the SAP particles are ‘full’ they cannot ‘easily’ absorb more liquid. Further, they do not give up their liquid contents easily either, particularly if they are isolated within a surrounding ‘dry’ environment. As explained above, once even partially swollen, their swelling rate decreases (see Zohuriaan-Mehr et al 2008), further liquid insults may be merely slightly absorbed. To some extent, further insults of liquid may bypass, at least in part, already swollen SAP particles.

When in vivo SAP particles are constantly surrounded by wet conditions, and diffusion can occur into and out of the particles. However, in in vitro conditions e.g. within disposable absorption articles for mammals e.g. humans, the particles are, in essence, isolated from one another, particularly when spherical or swollen. Indeed, no matter the initial shape, sphericity is increased in swollen SAP particles. Furthermore, typically in between the SAP particles is essentially dry, the liquid having been absorbed. Even when close-packed, swollen SAP particles may only touch one another at but a few points about their surface. Therefore, diffusion into and out of each SAP particle is low in such dry conditions. What has been absorbed, stays absorbed.

In effect, each SAP particle provides a limited volume which the present inventors have appreciated can be thought of as a sample chamber resistant to further ingress of liquid.

Even in bulk SAP, for example, one notional subdivision of SAP (e.g. with a mass) lies next to another subdivision of SAP (with the same predefined mass) and these are both swollen with sample liquid, then diffusion from one subdivision to the next is minimal, as there is no diffusion gradient. In effect, each subdivision of SAP provides a limited volume for absorption related to its mass within that subdivision. In effect, each subdivision forms a sample chamber, each sample chamber being adjacent to a neighbouring one.

This is easier to contemplate and, indeed, is more definitive where the SAP is actually physically subdivided into individual particles, preferably beads, more preferably spherical beads. Particles of other such shapes may be used such as, grains, rods, flakes and the like etc.

So, volume limitation to the assay is provided by notional, or actual, subdivision of SAP into particles. These subdivisions may be thought of as first sample chambers.

In one or more embodiments there may also be provided a second sample chamber within the physical configuration of an absorbent article or test sensor such as a test strip. Such a second sample chamber may have one or more impermeable (optionally flexible) walls and/or a closable entrance and so on. Such a second sample chamber may have one or more rigid walls. It may have a pre-defined sample chamber volume (e.g. of the second sample chamber volume). By providing a second (e.g. rigid walled, and/or well defined volume) sample chamber with at least one rigid wall, and preferably a rigid floor and rigid wall(s) and preferably also a rigid roof, the sample chamber volume is carefully defined. Nevertheless, the use of absorbent sensing material of the invention in combination with such a second sample chamber, eases the manufacturing challenges of such a chamber (e.g. in volume definition and/or surface properties and so on). Indeed, capillary surface size and/or properties may not be required.

After an initial insult, the liquid that has entered the SAP particles (or subdivisions of bulk SAP), notionally first sample chambers, remains there. It is not easily washed away or diluted even by further insults of liquid sample. This is particularly the case in absorbent articles for mammalian e.g. human wear, which are usually replaced every 2 to 6 hours, more usually every 2 to 3 hours. And even more the case in point-of-care or home test sensors where results are expected within seconds up to around 30 minutes to an hour.

Thus, by providing a limited volume of liquid sample in contact with a predetermined amount of reaction chemistry, a more accurate indication of the amount (e.g. the concentration) of analyte in the sample can be obtained. This insight offers some practical solutions to qualitative indications of disease and may offer opportunities for interrogation in a quantitative manner using the absorbent material and techniques of the invention.

In one or more embodiments, a main lateral dimension of dry SAP particles e.g. a length or diameter, may be ≤0.1 mm (≤100 micrometres, which would give a volume of a cube or sphere of or of the order of, 1 nL), or ≤0.05 mm (≤50 micrometres, which would give a volume of a cube or sphere of, or of the order of, 65 picolitres), or ≤0.01 mm (≤10 micrometres, which would give a volume of a cube or sphere of, or of the order of, 1 picolitre), or ≤0.005 mm (≤5 micrometres, which would give a volume of a cube or sphere of, or of the order of, 0.065 picolitres). At least e.g. 90% of the particles (or other proportion of the particles as disclosed elsewhere) may be of the stated dry volume e.g. within expected tolerances.

In one or more embodiments, a suitable dimension of swollen SAP particles into which a liquid sample has been absorbed, e.g. a length or diameter, may be ≤5 mm (≤5000 micrometres which would give a volume of a cube or sphere of, or of the order of, 6.5 microlitres), ≤1 mm (≤1000 micrometres which would give a volume of a cube or sphere of, or of the order of, 1 microlitres), or ≤0.5 mm (≤500 micrometres which would give a volume of a cube or sphere of, or of the order of, 65 nanolitres). The volume and/or other suitable dimension of the corresponding dry SAP particles which would absorb these amounts of liquid as a liquid sample of generally or substantially predetermined size is related to this swollen volume by the swelling ratio. The swelling ration depends on, amongst other things, the material from which the SAP is made. Examples of materials are shown in Table 1.

TABLE 1

| | Dry SAP Particle | Volume Range | |
|---|---|---|---|
| Material | Diameter Range | (pL to mL) | Material types |
| SAP | 10 μm-1 cm | picolitres to | Polyacrylates, |
| | | millilitres | Polyacrylamides, |
| | | | Polyvinyl alcohol |
| Nanoscale SAP | ≤100 μm | ≤0.05 pL | Polyacrylates, |
| | | | Polyacrylamides, |
| | | | Polyvinyl alcohol |

In one or more embodiments, the volume of the sample chamber provided by a single SAP particle is defined to be a particular size or in a particular range for example by defining a particular dimension e.g. diameter or other suitable dimension.

Examples are shown in table 2 in which example cube and sphere sizes are shown, and table 3 in which ranges of sphere sizes, here swollen sphere sizes Vmax, are shown:

TABLE 2

| | | | Side of Cube | | Diameter of Sphere | |
|---|---|---|---|---|---|---|
| Volume | | | with this volume | | with this volume | |
| pL | 10⁻¹² | L | 10 | μm | 12.4 | μm |
| nL | 10⁻⁹ | L | 100 | μm | 124 | μm |
| μL | 10⁻⁶ | L | 1 | mm | 1.24 | mm |
| mL | 10⁻³ | L | 1 | cm | 1.24 | cm |

TABLE 3

| Diameter | Vmax Range - | Vmax Range - | Vmax Range - |
|---|---|---|---|
| Range (nm) | Pico (pL) | Nano (nL) | Milli (mL) |
| 1- | 0.00005- | 0.05- | 50- |
| 10 | 0.05 | 50 | 50,000 |
| 10- | 0.05- | 50- | 50,000- |
| 100 | 50 | 50,000 | 50,000,000 |
| 100- | 50- | 50,000- | 50,000,000- |
| 1000 | 50,000 | 50,000,000 | 50,000,000,000 |

In one or more embodiments the purpose of selecting such dimension, in some embodiments at one end of the range e.g. a small dimension, for the majority (e.g. ≥50%, or ≥75%, or ≥90%, or ≥95%≥99%, in other words, preferably most) SAP particles is so that the volume of the sample chamber provided by each particle is more defined to be in a particular size for a selected application.

To explain, the actual volume of such a sample chamber is in effect limited by the size of the initial dry particle. Thus, the volume of a fully swollen SAP bead is closely based on its initial dimensions when dry, and so for spherical particles, its dry diameter, as well as to a lessor extent, the SAP type, the sample liquid, and to a even lessor extent the temperature, pressure, humidity, surrounding air flow etc. This mean that the maximum volume of the fully swollen particle (Vmax) depends on the SAP material, the swelling factor of the SAP material, the initial dry particle size, and environmental conditions (temperature, humidity, surrounding, air flow etc)

If it is assumed that the SAP bead has absorbed as much liquid as it can and is fully swollen, then the swelling volume (Vmax) represents the size of the sample chamber. In other words, the size of the sample chamber presented by the SAP particle has a volume equivalent to the amount of liquid that that SAP bead has absorbed. This in turn depends on various factors such as the type of SAP, the composition of the liquid, and the environmental conditions etc.

The benefits of this may be particularly useful where a very limited amount of sample chamber volume is available within one SAP particle, or a majority, or most, or all, where more than one is provided.

In one or more embodiments, which typically do not but may have the benefit of a second sample chamber as outlined above, individualised modified/preloaded SAP particles can provide a sample chamber of, in effect, a limited pre-definable volume.

The calculation for determining the diameter of a spherical SAP bead to achieve a certain volume can be done based on

V=1/6πD³F

where V is the volume, D is the diameter, F is the Swelling Factor, or swelling ratio, is the ratio of the volume of the fully swollen SAP bead to its dry volume, π (pi) is a mathematical constant with a value of approximately 3.14.

Swelling factor F is a dimensionless quantity that represents the amount of water absorbed per gram of dry SAP. The swelling factor F can vary depending on the specific type of SAP used and the conditions of the swelling. Therefore, in certain embodiments or scenarios, or for certain applications, it can be important to use the appropriate swelling ratio for the specific SAP material being used and/or specific conditions.

Table 4 shows various example of particle size (assuming as an example spherical particles, and various swelling factors F).

TABLE 4

| | | | | | | | | |
|---|---|---|---|---|---|---|---|---|
| Diameter D of | 5 | 12.407 | 50 | 100 | 200 | 124.07 | 12407 | 5000 |
| dry SAP bead | | | | | | | | |
| (μm) | | | | | | | | |
| Diameter of | 0.0005 | 0.0012407 | 0.005 | 0.01 | 0.02 | 0.012407 | 1.2407 | 0.5 |
| dry SAP bead | | | | | | | | |
| (cm) | | | | | | | | |
| Diameter of | 0.005 | 0.012407 | 0.05 | 0.1 | 0.2 | 0.12407 | 12.407 | 5 |
| dry SAP bead | | | | | | | | |
| (mm) | | | | | | | | |
| Radius (cm) | 0.00025 | 0.00062035 | 0.0025 | 0.005 | 0.01 | 0.0062035 | 0.62035 | 0.25 |
| Volume of a | 6.54E−11 | 1.00E−09 | 6.54E−08 | 5.24E−07 | 4.19E−06 | 1.00E−06 | 1.00E+00 | 6.54E−02 |
| dry SAP | | | | | | | | |
| sphere (cm3) | | | | | | | | |
| Volume of a | 6.54E−05 | 1.00E−03 | 6.54E−02 | 5.24E−01 | 4.19E+00 | 1.00E+00 | 1.00E+06 | 6.54E+04 |
| dry SAP | | | | | | | | |
| sphere (nL) | | | | | | | | |
| Volume of a | 6.54E−02 | 1.00E+00 | 6.54E+01 | 5.24E+02 | 4.19E+03 | 1.00E+03 | 1.00E+09 | 6.54E+07 |
| dry SAP | | | | | | | | |
| sphere (pL) | | | | | | | | |
| Swelling | 500 | 500 | 500 | 500 | 500 | 100 | 100 | 500 |
| factor, F | | | | | | | | |
| (ratio of water | | | | | | | | |
| in g absorbed | | | | | | | | |
| per g of dry | | | | | | | | |
| SAP) | | | | | | | | |
| volume of wet | 3.27E−08 | 5.00E−07 | 3.27E−05 | 2.62E−04 | 2.09E−03 | 1.00E−04 | 1.00E+02 | 3.27E+01 |
| SAP (Vmax) | | | | | | | | |
| (cm3) | | | | | | | | |
| Maximum | 3.27E+01 | 5.00E+02 | 3.27E+04 | 2.62E+05 | 2.09E+06 | 1.00E+05 | 1.00E+11 | 3.27E+10 |
| Sample Size | | | | | | | | |
| (pL) | | | | | | | | |

Therefore, assuming a consistent density and composition of material, a dry SAP bead with a diameter D of 12.4 microns has a volume of approximately 1 picolitres. If this has a swelling ratio of 500, then it absorbs (1×500) picolitres of liquid, so 500 picolitres. This is in effect the sample volume per particle.

Therefore, a dry SAP bead with a diameter D of 0.5 microns has a volume of approximately 0.065 picolitres. If this has a swelling ratio of 500, then it absorbs (0.065×500) picolitres of liquid, so 3270 picolitres or 3.27 nanolitres. This is in effect the sample volume per particle. Thus, SAP particles of various anticipated sample chamber volumes can be provided for different purposes, and/or different diagnostic techniques and/or different sample types.

Indeed, a dry SAP bead of 1.24 cm could provide absorption of 100 cm\hat{ }3 if the swelling factor is 100.

One or more embodiments of this invention relate to medical diagnostics, specifically to the screening of large populations of infants for indicators of chronic diseases, for example diabetes. Such screening enables prompt medical intervention and the avoidance of complications resulting from late diagnosis.

Typically, chronic conditions are diagnosed using measurements of the concentration of analytes in biofluids. In the case of diabetes, the most relevant analyte is glucose in blood or urine. Such measurements may be made using either disposable test strips or laboratory analysers. Disposable test strips can measure glucose in both blood and urine.

To measure glucose in blood it is necessary to extract a blood sample and introduce it to the test strip or laboratory analyser. This involves puncturing the skin, which causes pain, rendering such an approach unsuitable for routine measurements in populations of infants, the vast majority of whom will not display any symptoms. Using a laboratory analyser has the additional disadvantage that the infant or the sample must be transported to a central analysis facility.

The measurement of glucose in urine does not result in any pain, but in infants who are not potty trained it is extremely challenging to obtain a suitable sample of urine for measurement in the home using a disposable test strip. If a laboratory analyser is used the same practical limitations as noted above apply.

Diabetes screening shows 60% of under 2 s are already in diabetic ketoacidosis at the time of diagnosis. Some subjects present with ketoacidosis-related cerebral oedema at diagnosis leading to permanent, severe disability requiring wheelchairs and residential care.

In one or more embodiments of the invention, when an infant wets their nappy the urine contacts absorbent sensing material, and the parent or carer gets a clear indication of the result. The absorbent sensing material may be configured to display a ‘traffic light’ type output for one or more analytes, indicating that the urine is either Normal (in which case no action is required), Alert (in which case a Health Care Professional should be consulted for follow-up tests) and Alarm (in which case immediate medical attention should be sought).

One embodiment of the invention is a disposable wearable article such as a nappy (diaper) liner, or a nappy (diaper), containing absorbent sensing material capable of detecting and indicating a level of analyte e.g. glucose and, optionally, other analytes in urine based on one or more sensing element(s) which indicate, e.g. change colour or otherwise provide an indication, when in contact with the relevant analyte.

A key challenge to overcome is discriminating between a small amount of more concentrated sample fluid e.g. urine and a larger amount of less concentrated sample fluid e.g. urine, both of which may contain the same absolute quantity of analyte. This challenge is addressed using a fluid capture system such as the absorbent sensing material which, as explained above, over the time scale of use is capable of receiving a finite amount of urine. In one or more embodiments, the absorbent sensing material is located between the source of the urine and an impermeable layer, typically located beneath the absorbent matrix. The impermeable layer may prevent further ingress of fluid once the absorbent sensing material is saturated. The capacity of each part (e.g. particles) of the absorbent sensing material is preferably configured such that it is saturated with a limited urine volume. Due to the finite fluid capacity of the absorbent sensing material, the reaction components come into contact with a more defined amount of analyte.

Each SAP particle is in one sense capable of receiving only a more defined volume of urine. Therefore, the amount of analyte that is delivered to each SAP particle is more closely proportional to the concentration in the bulk fluid. Once the SAP particle is full or partially full, it is configured to resist further ingress of fluid. Without wishing to be bound by theory, it is thought this may be due to a reduction in swelling rate over time.

In one or more embodiments which effect a colour change, the reaction components consist of a sensing chemistry immobilised internally. For example, in the case of glucose, a two-stage dual enzyme system may be used in which Glucose Oxidase (GOD) and Horseradish Peroxidase (HRP) are provided. In the first stage Glucose Oxidase (GOD) catalyses the reaction of glucose to gluconic acid and hydrogen peroxide. In the second stage, Horseradish Peroxidase (HRP) catalyses the reaction of peroxide with the initially colourless chromogen (dye precursor) and a colour develops. The colour will depend on the chromogen selected. For example, with potassium iodide, green to brown colours are accessible. With 3,3′,5,5′-tetramethylbenzidine (TMB) blue colours are accessible. Other colours are accessible using different chromogens (see FIG. 8 and Examples below).

Various implementations of the Normal, Alert and Alarm levels are possible. Each level is indicated by a separate sensing compartment containing SAP particles, one each for Normal, Alert and Alarm. These may be differentiated on the basis of colour, achieved by selecting a different coloured chromophore for each sensing compartment. Initially, the contents (SAP particles) of all three sensing compartments are white. On contact with normal urine, the Normal sensing SAP particles would turn green and the Alert and Alarm sensing elements would remain white. On contact with urine containing an intermediate concentration of glucose, the Normal SAP particles would turn green, the Alert SAP particles would turn orange and the Alarm SAP particles would remain white. On contact with urine containing a high concentration of glucose, the Normal SAP particles would turn green, the Alert SAP particles would turn orange and the Alarm SAP particles would turn red.

An alternative implementation is possible, in which the same chromogen is used in the Normal, Alert and Alarm sensing elements. There are two variants. In the first, each group of SAP particles contains increasing loadings of chromogen, such that a normal glucose concentration is indicated by one light coloured e.g. blue dot, an intermediate Alert concentration is indicated by one light coloured, e.g. blue, dot and a coloured, e.g. blue, dot of medium intensity, while a higher Alarm concentration is indicated by one light coloured, e.g. blue, dot, one coloured, e.g. blue, dot of medium intensity and a further coloured, e.g. blue, dot of greater intensity.

In the second variant, the same level of the same type of chromogen would be present in each group of SAP particles, with the sensing performance of each differentiated on the basis of the enzyme system and loadings, such that the sensing elements' SAP particles in each compartment each reach the same colour end point, such that a normal glucose concentration is indicated by one blue dot, an intermediate Alert concentration is indicated by two blue dots, while a higher Alarm concentration is indicated by three blue dots.

In one or more embodiments, at least two groups of SAP particles are provided, at least one group configured to be indicative of a first level of analyte, and a second group configured to be indicative of a second level of analyte.

### Preparation of Sensing Elements

This section describes the preparation of sensing elements (SAP beads).

### Example 1

SAP beads were prepared using two-stage dip process in which SAP beads were first impregnated with enzymes, a dye fixer and a colour enhancer, then impregnated with the chromogen and a film-forming agent.

Specifically, SAP beads were dipped into Solution 1, a 25% ethanol solution buffered to pH 4.8 with citrate buffer, containing the enzymes GOD and HRP, the dye fixer Gantrez AN-139, and the colour enhancer polyyinylpyrrolidone. The SAP beads were then allowed to dry.

The SAP beads were then dipped into Solution 2, consisting of the chromogen TMB and a film forming agent ethyl cellulose in chloroform. The SAP beads were then dried.

The steps above were performed three times with increasing chromogen concentration to create the Normal, Alert and Alarm sensing elements.

The variant described herein is that in which the same chromogen is used in the Normal, Alert and Alarm sensing elements. Each of the three portions of sensing elements contains increasing loadings of the same chromogen, TMB.

### Example 2

Absorbent polymer (SAP) beads were stably loaded with reagents that would react with a glucose solution to produce a visually detectable colour change. The ability of loaded beads to react with glucose, even after the beads had been dried to their native state has been demonstrated. In their native state they are typically ≤0.5% water by weight.

A pre-existing commercial assay was loaded onto the beads using a reaction mix containing an enzyme and a reagent which produces a visible colour change in the presence of the product of the reaction between the enzyme and glucose.

The stability of the loaded beads was tested for freezing at −80° C. and subsequently for loaded beads that underwent freeze-drying or thermal drying. The number of beads for each assay and the volume of reagent required was determined.

As will be understood by those skilled in the art, the sensitivity of this approach can be demonstrated using a number of glucose concentrations (relatively low to relatively high) to transect the physiological glucose concentration range. Furthermore, the beads remain active after drying. Furthermore, long-term stability and potential for suitability for long term storage is indicated after early stage accelerated stability testing.

Absorbent beads in the form of a super absorbent polymer consisting of sodium polyacrylate was used. This has no known toxicity or hazard.

A glucose assay kit (colorimetric/fluorometric) was used e.g. from Abcam (product ab65333). This is a well-characterized, simple and rapid assay used to quantify glucose levels in biological samples. The supplied enzyme mix oxidises glucose, generating a product that reacts with a dye which generates a red colour which is visible to the naked eye.

Glucose Assay Kit ab65333 is a rapid, simple and sensitive assay used to quantify glucose levels in biological samples such as serum, plasma, and other body fluids, food, growth medium, etc. In the glucose assay protocol, the glucose enzyme mix oxidizes glucose to generate a product which reacts with a dye to generate color (λ=570 nm) and fluorescence (Ex/Em=535/587 nm). The generated colour and fluorescence is proportionally to the amount of glucose. The kit detects glucose in the range 1-10000 μM. The detection regime is shown in FIG. 8 using

The assay is designed for use with a plate reader to detect and quantify colour or fluorescence. This study was adapted to function with the beads in the in vitro environment selected to provide a visual assessment of an end point.

To compensate for the absorbent nature of the beads, the volumes used were adjusted using additional buffer, while at the same time retaining the recommended proportions between the reaction mix (enzyme and probe) with the glucose solution.

Coated beads were freeze-dried using a CHRIST ALPHA1-2 LD_{Plus} freeze drier, according the manufacturer's instructions.

For the purposes of these experiments, it was not possible to measure the intensity of the colour of the reactions using absorbance (at 570 nm) because of the size and characteristics of the beads. Therefore, descriptions of the observations were used along with contemporary photographs shown in FIGS. 1 to 5.

a. Bead Number and Reagent Volumes

For the purposes of the glucose assay using beads (here spherical beads), an optimal number of beads and a volume of reagents that was in proportion to the requirements of the assay kit was determined, on a scale that did not consume the reagents but was visibly detected.

Initial experiments using sequential addition of small volumes of water indicated that if 10 beads were used, a volume of up to 250 μl of reagent could be completely absorbed without residual, non-absorbed water remaining on the surface of the retaining vessel (described as a boat). Where volumes of up to 500 μl were used, the water was not fully absorbed demonstrating that the SAP beads have a limit in their swelling capacity. In this experiment, subsequently, volumes of no greater than 250 μl were used. A comparison between beads with 250 μl water and without water are shown in FIG. 1. FIG. 1 shows SAP beads when initially dry (lower) and when completely swollen (upper). The initially dry SAP beads therefore present a limited volume to a sample (a sample volume).

The initially dry SAP beads are much smaller in diameter than the water swollen SAP beads (now hydrogel beads).

In Experiment 1 (see FIG. 2), in a first step, the beads were loaded with reaction mix. This was the initial step in demonstrating the beads could be loaded with the reaction mix in solution, dried, and retain the ability to detect glucose in solution. Thus, dry SAP beads pre-loaded with one or more reaction components can be prepared.

Two sets of beads were prepared, a test set imbibed with 250 μl of reaction mix (described as test beads (right)) and a control set coated with assay buffer only (described as control 1 beads (left)). The beads readily absorbed the reagents after 30 min incubation in the dark at room temperature. Addition of 250 μl of 1 mM glucose solution to both test and control beads, followed by 30 min incubation at 37° C. in the dark produced a strong reaction in the test beads (pink/brown colour), while the control beads remained colourless (see FIG. 2). FIG. 2 shows groups of 10 SAP beads in Experiment 1 which have been (hydrated) pre-loaded with buffer only (left image) and then exposed to glucose solution for 30 minutes at 37° C., or (hydrated) pre-loaded with liquid reaction mix (right), and then exposed to glucose solution for 30 minutes at 37° C. The beads on the left remain colourless and a clear colour change to pink brown is visible on the right.

This result indicated that the beads could not only absorb the reaction mix, but also that the reagents remained functional after absorption when wet. Wet SAP beads, in other words liquid swollen beads of hydrogel, are, however, impractical, and may not offer solutions over the time frame of any desired tests.

c. Effect of Heat-Drying of Loaded Beads on Assay Function

In Experiment 2 (see FIG. 3), it was determined whether coated beads could be dried and stored for future use, to provide pre-loaded dry SAP particles without losing their reactivity with glucose. Various methods can be used. One is heat-drying.

Test beads (treated with reaction mix) and control beads (control 1) treated with assay buffer only were prepared as described above, along with a second control (control 2) treated with reaction mix to give three sets of beads in total. Based on an optimisation experiment (not shown), the volume of reaction mix used was reduced to 200 μl.

Following treating, the beads were transferred to individual tubes and placed in a rotating heating block set to 35° C. for 18 hours in the dark, after which the beads were reduced in size but not dry. The beads required a total incubation at 35° C., with rotation, of 48 hours in the dark, by which time the beads were further reduced in size, with test and control 2 beads slightly pink in colour. The beads were sticky and difficult to manipulate but decanted onto weighing boats for assay.

The assay was performed by adding 200 μl of 1 mM glucose solution to control 1 and test beads, or assay buffer to control 2 beads. The results indicate a relatively high background colour in the absence of glucose (control 2), but the test bead positivity is sufficiently strong to differentiate it from this control (see FIG. 3). FIG. 3 and FIG. 4 show perspective view of groups of 10 SAP beads in Experiments 2 and 3 showing respectively heat-dried beads (FIG. 3) and freeze-dried beads (FIG. 4):
control 1 (top) treated with buffer, dried, then incubated with glucose solution for 30 minutes at 37° C., no colour is seen:
control 2 (middle) treated with liquid reaction mix, dried, then incubated with buffer for 30 minutes at 37° C., a light pink colour is seen;
test beads (lower) treated with liquid reaction mix, dried, then and incubated with glucose solution for 30 minutes at 37° C., a deeper purple colour is seen.

This result indicated that despite heating at 35° C. for −48 hours, the test beads retained their reactivity with glucose.

d. Effect of Freeze-Drying of Loaded Beads on Assay Function.

The second method selected for drying the beads was freeze drying. This presented a specific difficulty in that the initial step requires freezing of the coated beads at −80° C.; freezing of the beads is not recommended by the manufacturer. However, a pilot study revealed that freezing and thawing of beads loaded with 200 μl of reaction mix did not cause visible disruption of the bead structure or cause loss of activity of the reaction mix (not shown).

In Experiment 3 (FIG. 4), three sets of beads test (reaction mix), control 1 (buffer) and control 2 (reaction mix) were set up (as described for heat drying), transferred to cryovials and frozen at −80° C. The freeze dryer was set up according to the manufacturer's instructions, the beads removed from the freezer and freeze dried for 5 hours.

When decanted onto boats, for each treatment all beads were present, visually undamaged, rolled easily and did not aggregate (unlike the heat-dried beads). The beads were small, similar in size to native beads, but the appearance of the beads had changed from the fresh, dry, native beads. They were opaque, not as smooth in appearance, and control 2 and test beads had a weak pink appearance.

Once dried, the beads were then treated with buffer (control 1) or glucose in buffer (test, control 2), as described above. Following incubation at 37° C. for 30 mins in the dark, control 1 beads were colourless, control 2 had a light pink background, while test beads were a deeper pinkish brown colour, with some white patches, which is indicative of a strongly positive result (see FIG. 4) as also described above.

Of the two approaches taken for drying the beads, freeze-drying was a much more effective method than heat-drying. The results show that modified or pre-loaded beads retain their structure and function after both freezing at −80° C. and the freeze-drying process. Thus, pre-loaded dried beads can be formed.

e. Use of Water in the Reaction Mix

Test and control beads that were freeze dried had a change in appearance (described above). It was thought that the surface changes and opacity may be due to the accumulation of buffer salts on the beads following drying. This may have an adverse effect on readability or interpretation so water was substituted for buffer in the reaction mix; glucose would be delivered in buffer, as previously.

In experiment 5, the beads were treated with water and glucose as described above. FIG. 5 shows groups of 10 SAP beads in Experiment 4 showing respectively:
control 1 (top) treated with liquid water, freeze-dried, then incubated with glucose solution for 30 minutes at 37° C., no colour is seen;
control 2 (middle) treated with reaction mix, freeze-dried, then incubated with buffer for 30 minutes at 37° C., almost no colour is seen.
test beads (lower) treated with reaction mix, freeze-dried, then and incubated with glucose solution for 30 minutes at 37° C., a purple colour is seen.

The water-substituted reaction mix seemed to be absorbed more readily by the beads than with buffer. Further work with freeze drying demonstrated no change of appearance of the beads, potentially indicating that buffer salts were present previously and using water prevented them from accumulating. Consequently, water, as a substitute for buffer in reaction mix is preferred. The use of water in the reaction mix did not affect the reaction of treated beads with glucose (see FIG. 5). Following incubation of 30 mins at 37° C., it can be seen that control 1 beads (top) are colourless and control 2 beads are very light pink (middle). Test beads showed a clear colour change (bottom).

The sensitivity of the assay, relative to a range of concentrations of glucose will provide a different depth of colour, or using Benedict's solution, a range of colours.

The retention of activity of treated, freeze-dried, dry SAP beads over time (“shelf-life”) is much longer than for undried loaded, swollen hydrogel beads.

It has been demonstrated that:
The beads can absorb the glucose assay reaction mix.
The reaction mix retains activity after absorption.
Heat-dried test beads retain their reactivity with glucose.
Freeze-dried test beads retain their reactivity with glucose.
Freeze-drying was a much more effective method than heat-drying.
Test beads retain their structure and function after undergoing both freezing at −80° C. and the freeze-drying process.
Freeze-drying changes the appearance of test beads when buffer is used in the reaction mix and this may be due to buffer salts.
Freeze-drying appearance changes are not observed where water is substituted for buffer in the reaction mix.
Water should be used as a substitute for buffer in reaction mix in future experiments to avoid background colour that could represent a false positive.

Preferably, buffer is not used in the reaction mix. Preferably water is used, particularly if freeze-drying is to be used.

It has been shown that partially swollen beads containing reaction mix can swell further upon insult with analyte. The limited mass of each particle provides a limit to the total amount that can be absorbed. It has been shown that swollen beads containing reaction mix can be dried and retain activity when rewetted. Use of dry superabsorbent polymer particles containing dry reaction component(s) in the detection of analytes in vitro liquids has been demonstrated.

The invention provides a method for manufacturing absorbent articles or test sensors and a method for carrying out detection of analyte using the absorbent articles or test sensors as shown in FIG. 7. In step 10, initially dry SAP/SAP particles are pre-loaded with at least one wet reaction component e.g. in solution, to cause the SAP particles to form swollen hydrogel particles.

In step 20, primarily water is driven off from the SAP/SAP particles by drying these e.g. by heat and/or freeze-drying e.g. until a desired residual water content is achieved.

In step 30, the now dry SAP/SAP particles pre-loaded with at least one reaction component are added to an absorptive item, e.g. a disposable hygiene item or to a test sensor, during manufacture of same.

Finally, in step 40, the dry pre-loaded SAP/SAP particles are used as an indicator or diagnostic of an analyte in sample liquid, by exposing this/these to liquid, and detecting and indicating the presence or concentration of the analyte.

Thus, the use of dry superabsorbent polymer (SAP)/SAP particles containing dry reaction component(s), in other words dry modified (preloaded) SAP can be extended to other detection scenarios, and interrogation techniques for example those that require a limited sample chamber volume (e.g. a ‘second’ sample chamber in addition to the notional sample chamber provided by the SAP).

The present inventors have appreciated a simple solution for indicating or monitoring a disease state using a body fluid or, more generally, for indicating the presence of an analyte in a sample liquid. In one or more embodiments, the present invention does not use a separate sensor. In one or more embodiments, the present invention does not (initially at least) use hydrogel (in the sense of wet SAP). Where SAP particles such as beads are used, their shape may be spherical, cylindrical, irregular, regular, cubic, flakes, prismatic, fibre-like, hollow, or any 2 or 3-dimensional structure. In one or more embodiments, SAP particles are positioned within an article e.g. a wearable article, which has a window e.g. a clear, preferably colourless, panel for observing the SAP particles (e.g. before and/or during and/or after insult with a sample liquid). In other words, the window may be on the inside (hidden during use) or outside (visible during use) of the article.

The manufacture of articles, especially wearable articles is not complicated. In some embodiments, SAP particles are included in such articles as before, however, the SAP particles have undergone a previous manufacturing step, namely impregnation or pre-loading with one or more reagents (reaction components) in one or more reagent mixes and then drying as usual, preferably, freeze-drying.

The now pre-loaded SAP (e.g. beads) swollen with wet reaction mix may be dried by any suitable method. For example, heating in an oven, optionally on a conveyor passing through an oven, or by freeze drying or freezer. The residual moisture content in dry SAP for use in one or more embodiments of the invention is typically 0.5% to 15% by weight, or 1% to 10% by weight, or less than 15% by weight or less than 10% by weight or less than 5% by weight. The residual moisture content may be determined by EDANA (European Disposables and Non-Woven Association) recommended test method No. NWSP 230.0.R2 ‘Moisture Content’.

The SAP particle size may be determined by means of EDANA (European Disposables and Non-Woven Association) recommended test method No. NWSP 220.0.R2 ‘Particle Size Distribution’. The swelling capacity of the SAP e.g. of particles may be determined by EDANA recommended test method No. NSWP 240.02.R2 ‘Free Swell Capacity’. The fluid retention capacity may be determined by the EDANA recommended test method No. NWSP 241.0.R2. Other parameters may be determined from the EDANA test methods or adapted test methods if required e.g. swelling rate. Other recognised standard test methods may be used.

The art discusses how to make SAP beads but not how to use SAP beads in practical, commercial methods for analyte detection in body liquids or, indeed, in other liquids. The use of SAP beads as an effective sample chamber has not previously been discussed in the art. In one or more embodiments, dry SAP beads are used in a process for detection of an analyte. In one or more embodiments of the invention, the SAP itself is not responsive to specific molecules. Further, in one or more embodiments, the SAP is not required to give back the imbibed solution or maintain the imbibed solution around it to allow diffusion. In one or more embodiments of the invention, the detection (transduction) mechanism is independent of the carrier membrane i.e. independent of the SAP. In one or more embodiments, the present invention aims to provide a simple to use, and/or simple to manufacture, and/or simple to implement, and/or simple to read system for detecting analytes.

For simplicity, all the absorbent material in an absorbent article or test strip for other interrogation techniques may be absorbent sensing material according to the present invention. However, only a portion may be. For example, an absorbent article may comprise absorbent sensing material comprising pre-loaded SAP in a portion of the absorbent article e.g. in a central portion, or in an indicator portion which may be at the front or rear or base of an article, and the remaining absorbent material may be regular dry SAP particles that do not have a sensing capability. A separate fluid compartment may be provided for absorbent sensing material, separate from that containing regular SAP particles. Different compartments may be provided with absorbent sensing material of the present invention, with different sensing capabilities e.g. different reagent components for different assays (e.g. urine and ketone) or of different sensitivities, so a higher concentration of analyte is required before a signalling component (e.g. a colour change is seen). It will be understood by those skilled in the art that the absorbent material may be used in a test strip or other sensors technologies for interrogation by visual or other methods.

The SAP particles are of limited volume and solve the problem of requiring a limited volume in a different way—the internal volume or rather swelling capacity of each SAP bead limits the volume of sample that can be interrogated in each particle, particularly in vitro, where the presence of other surrounding SAP particles tend to main a dry environment outside each SAP particle. Whilst use of dry SAP particles is known in absorbent articles, its use as a medium for interrogating sample liquids, particularly for interrogating sample liquids in absorbent articles or other sensors technologies, is not known. This ability to combine the use of SAP as an absorbent and as a carrier for one or more dry detection and signalling components, facilitates very simple use. Typically, the signalling component is observable by eye, although other measurable or observable regimes can be used.

This ability to combine the use of SAP as an absorbent and as a carrier of reagents also facilitates very simple manufacture of absorbent articles. The dry SAP particles are pre-loaded with reagent mix comprising one or more dry detection and signalling components (in the absence of any substrates or analytes being detected) and then dried as usual. These may be air-dried, heat-dried or freeze-dried or use any suitable drying method to form absorbent sensing material that can be used in multiple applications. The absorbent material in the form of pre-loaded SAP, preferably SAP particles, can therefore, as is usual now, be manufactured separately in advance of the absorbent articles. The manufacturing process of absorbent articles does not need to be charged, save that a viewing window may be provided, preferably on the exterior to the absorbent article. The absorbent article may have a permeable interior (in wearable articles skin facing) layer, and an impermeable exterior (in wearable articles outer) layer. Indeed, the absorbent material with sensing capability may be used in many different applications, both in human and animal healthcare and in liquid analysis applications.

Indeed, the absorbent material of the invention e.g. in the form of SAP particles may be manufactured in advance ready for use in other sensors technologies, other than absorbent articles. It will be further clear to those skilled in the art that the invention is not limited to sensing elements which are based on colorimetric detection.

It will be clear to those skilled in the art from this disclosure that the invention could be applied to other analytes besides glucose and other disease states besides diabetes. Staying with diabetes as an example, a similar dual enzyme detection method may be used to detect ketones in urine, indicative of diabetic ketoacidosis. Broadening to other disease states, protein in urine is indicative of kidney inflammation, nitrite and white blood cells are indicative of bacterial infection, bilirubin and urobilinogen are indicative of liver damage. Each analyte may be detected in a manner analogous to that described for glucose above. Multiple analytes may be detected simultaneously by combining a series of sensing elements.

It will also be clear to those skilled in the art that the invention is not limited to the detection of analytes in urine. It is appropriate to any biofluid, for example it could also be applied in a transdermal patch in which interstitial fluid is drawn into the fluid capture system, or indeed a dry tissue for blowing a nose or wiping away tears, or a nasal or throat swab for sampling nasal or throat fluids. The absorbent material may, in these examples, be in the form of fibres

In one or more embodiments, the present invention at least partially addresses one or more problems of the art:
variability of concentration of analyte in sample liquid, and/or a variable flow, e.g. of urine, and/or a variable contact time do not render the test invalid;
additional insults of bodily fluid do not render the test invalid;
sample fluid discharged into an absorbent matrix may not affect the remaining liquid presented to any test element;
SAP or SAP particles or sensors using SAP or SAP particles attached to diapers may more easily limit sample volume. These may also address cost and complexity of manufacture and use;
SAP e.g. SAP particles do not break and/or are comfortable to wear, they also relatively cheap;
detection of enzyme substrates is possible;
a diffusion gradient is not required;
dry SAP sensing systems are stable over the time periods for long term storage required for commercial products;
a controlled sample volume, e.g. a ‘second’ sample chamber in addition to the notional chamber(s) provided by SAP, can be more easily manufactured.

In one or more embodiments, the invention provides, within the dry superabsorbent polymer, at least one reaction component for detecting and signalling the presence of analyte, and/or, in some embodiments, for detecting and signalling the concentration, e.g. the relative level of concentration of analyte, and/or, in some embodiments, for detecting and signalling the relative quantity of analyte; wherein upon insult of sample liquid, the dry super absorbent polymer absorbs sample liquid, the dry reaction component, dissolves, preferably within the SAP, and if analyte is present in the sample liquid, its presence is detected and signalled within the SAP itself.

1. An absorbent sensing material for interrogation of sample liquid to detect analyte,
comprising:
a modified dry superabsorbent polymer (SAP) configured to receive a sample liquid;
the modified dry superabsorbent polymer comprising a dry superabsorbent polymer and at least one dry reaction component for detecting and for signalling the presence of analyte;
wherein, upon insult of sample liquid, the modified dry superabsorbent polymer absorbs sample liquid, the at least one dry reaction component dissolves and, if analyte is present in the sample liquid, its presence is detected and signalled.

2. An absorbent sensing material according to claim 1 comprising: at least 50%, or at least 75%, or at least 90%, or at least 95%, by weight of modified dry superabsorbent polymer comprising the at least one dry reaction component.

3. An absorbent sensing material according to claim 1 in which the modified dry superabsorbent polymer material comprises at least 95% by weight, or at least 97% by weight of dry superabsorbent polymer and up to 5% by weight, or up to 3% by weight, of the at least one dry reaction component(s).

4. An absorbent sensing material according to claim 1 in which modified dry superabsorbent polymer is configured to receive a discrete amount of a sample liquid.

5. An absorbent sensing material according to claim 1 in which the modified dry superabsorbent polymer provides a sample chamber of at least one of: limited mass, limited volume.

6. (canceled)

7. (canceled)

8. (canceled)

9. An absorbent sensing material according to any of claim 1 in which the modified dry superabsorbent polymer comprises particles.

10. (canceled)

11. An absorbent sensing material according to claim 9 in which at least 90% by weight of the particles are within a range selected from one of the following: ≤±10%≤±5%≤±2% of the 0 dry weight of the particles.

12. An absorbent sensing material according to claim 9 in which the particles are within a range of ≤+/−10% the average size particles.

13. An absorbent sensing material according to claim 12 in which, when dry, the particles have a main dimension selected from of: ≤100 nanometres, 100 to 1000 nanometres, 100 to 5000 nanometres, 5 to 1000 microns, 5 to 500 microns, 5 to 250 microns, 500 microns, ≤0.1 mm, 0.05 mm, ≤0.01 mm≤0.005 mm 0.5 to 5 mm, from 1 mm to 4 mm, from 0.5 mm to 3 mm, from 0.5 mm to 2 mm, from 1 mm to 2 mm.

14. An absorbent sensing material according to claim 1 in which the at least one dry reaction component comprises:
at least one dry transducing component for detecting an analyte;
and,
at least one dry signalling component responsive to the detection of analyte for signalling the presence of analyte.

15. An absorbent sensing material according to claim 1 in which the at least one reaction component comprises at least one of
an electrochemical assay;
a colorimetric assay;
a nitrate assay;
a ketone assay;
a glucose assay;
a bilirubin assay;
a component capable of photometric detection;
a component capable of spectrometric detection.

16. An absorbent sensing material according to claim 1 in which the at least one reaction component comprises a colorimetric assay comprising a chromogen.

17. (canceled)

18. (canceled)

19. An absorbent sensing material according to claim 1 in which the modified dry superabsorbent polymer comprises at least 50% or at least 75% or at least 90% by weight of at least one of:
polymerised acrylic acid, salts of polymerised acrylic acid, sodium polyacrylate, polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile.

20. An absorbent sensing material according to claim 19 in which the superabsorbent polymer comprises at least 50% or at least 75% or at least 90% by weight of at least one of: polymerised acrylic acid or salts thereof, sodium polyacrylate, polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile.

21. A disposable absorbent article for sample fluid interrogation comprising an absorbent core, at least a portion of the absorbent core comprising absorbent sensing material for interrogation of sample liquid to detect analyte, the absorbent sensing material comprising:
a modified dry superabsorbent polymer (SAP) configured to receive a sample liquid;
the modified dry superabsorbent polymer comprising a dry superabsorbent polymer and at least one dry reaction component for detecting and for signalling the presence of analyte;
wherein, upon insult of sample liquid, the modified dry superabsorbent polymer absorbs sample liquid, the at least one dry reaction component dissolves and, if analyte is present in the sample liquid, its presence is detected and signalled.

22. A disposable absorbent article according to claim 21 in which at least a portion of the modified dry superabsorbent polymer of the absorbent material comprises at least one of: superabsorbent polymer particles, superabsorbent polymer fibres.

23. (canceled)

24. (canceled)

25. A disposable absorbent article according to claim 21 in which the absorbent sensing material is provided within a compartment of limited volume, having at least one impervious wall to resist further ingress of sample liquid once full.

26. A disposable absorbent article according to claim 21 in which at least two groups of modified dry superabsorbent polymer particles are provided, at least one group configured to be indicative of a first level of analyte, and a second group configured to be indicative of a second level of analyte.

27. Use of an absorbent sensing material according to claim 1 for detecting and for signalling the presence of one or more analytes in a sample liquid—in which the modified dry superabsorbent polymer is used as a sample chamber to absorb a predetermined amount of the at least one reaction component.

28. (canceled)

29. (canceled)

30. A method of manufacturing an absorbent sensing material for interrogation of sample liquid to detect analyte, the absorbent sensing material comprising: a modified dry superabsorbent polymer configured to receive a sample liquid; the modified dry superabsorbent polymer comprising a dry superabsorbent polymer and at least one dry reaction component for detecting and for signalling the presence of analyte; wherein, upon insult of sample liquid, the modified dry superabsorbent polymer absorbs sample liquid, the at least one dry reaction component dissolves and, if analyte is present in the sample liquid, its presence is detected and signalled, the method comprising:
a. preparing superabsorbent polymer particles;
b. drying the superabsorbent polymer particles;
c. in the absence of an analyte, introducing at least one wet reaction component for detecting and signalling the presence of the analyte, to the dry superabsorbent polymer particles;
d. drying the modified superabsorbent polymer particles to form modified dry superabsorbent polymer particles pre-loaded with the at least one dry reaction component for detecting and signalling the presence of an analyte.

31. (canceled)

32. A process for detecting analyte in a fluid comprising:
a) providing absorbent sensing material according for interrogation of sample liquid to detect analyte, the absorbent sensing material comprising: a modified dry superabsorbent polymer (SAP) configured to receive a sample liquid; the modified dry superabsorbent polymer comprising a dry superabsorbent polymer and at least one dry reaction component for detecting and for signalling the presence of analyte; wherein, upon insult of sample liquid, the modified dry superabsorbent polymer absorbs sample liquid, the at least one dry reaction component dissolves and, if analyte is present in the sample liquid, its presence is detected and signalled;
b) insulting the absorbent sensing material comprising modified dry superabsorbent polymer, with sample liquid
c) optionally, re-insulting the absorbent sensing material with sample liquid;
d) at least partially rehydrating the modified dry superabsorbent polymer;
e) at least partially rehydrating the at least one reaction component to detect and signal the presence and/or concentration of analyte in the sample liquid;
f) detecting and signalling the presence of analyte in the sample liquid.

33. An absorbent material to claim 1 in which at least two groups of modified dry superabsorbent polymer particles are provided, at least one group configured to be indicative of a first level of analyte, and a second group configured to be indicative of a second level of analyte.
